(19) United States
(12) Patent Application Publication
(10) Pub. No.: US 2024/0293788 A1
(43) Pub. Date: Sep. 5, 2024

(54) DEVICE AND PROCESS SET-UP FOR EQUILIBRIUM REACTIONS

(71) Applicant: Universiteit Antwerpen, Antwerpen (BE)

(72) Inventors: Serge Tavernier M.F., Antwerpen (BE); Yorick Clauwers, Antwerpen (BE); Toon Bouwen, Antwerpen (BE)

(73) Assignee: Universiteit Antwerpen, Antwerpen (BE)

(21) Appl. No.: 18/565,297

(22) PCT Filed: Jun. 9, 2022
(86) PCT No.: PCT/EP22/65754

§ 371 Date: Nov. 29, 2023

(30) Foreign Application Priority Data
Jun. 10, 2021 (EP) 21178858.3

(51) Int. Cl. B01J 8/10; B01J 8/08; C12M 1/40

(52) U.S. Cl. CPC B01J 8/10; B01J 8/082; B01J 8/085; B01J 8/087; C12M 21/18; B01J 2208/00628

(57) ABSTRACT

The present invention relates to devices for maintaining a state of equilibrium, and process set-ups to perform equilibrium reactions using heterogeneous catalysts, in particular said reactions are equilibrium reactions where at least one reaction product is volatile. The invention is especially interesting for enzymatic reactions using immobilized enzymes, although it is also useful for pure chemical reactions. The invention is characterized by a combination of non-impact mixing of components and volatilization and/or removal of excess molecules, thereby maintaining a state of equilibrium or driving an equilibrium reaction. The present invention also relates to devices and/or process set-ups and materials made using said processes.

### FIELD OF THE INVENTION

The present invention relates to devices for maintaining a state of equilibrium, and process set-ups to perform equilibrium reactions using heterogeneous catalysts, in particular said reactions are equilibrium reactions where at least one reaction product is volatile. The invention is especially interesting for enzymatic reactions using immobilized enzymes, although it is also useful for pure chemical reactions. The invention is characterized by a combination of non-impact mixing of components and volatilization and/or removal of excess molecules, thereby maintaining a state of equilibrium or driving an equilibrium reaction. The present invention also relates to devices and/or process set-ups and materials made using said processes.

### BACKGROUND TO THE INVENTION

The present invention addresses major problems encountered in a lot of chemical and enzymatic reactions making use of catalysts. First at all, it is known to experts in the field that when using heterogeneous catalysts, the surface may be destructed during stirring or mixing of the reaction medium, thereby reducing their catalytic activity. Moreover, immobilization of catalysts, such as enzymes, is an essential step towards industrialization of (enzymatic) processes for the production of mid value to low value (commodity) products as recycling of the catalysts (e.g. enzymes) is essential for cost reasons.

However, immobilized catalysts and in particular enzymes are very sensitive to mechanical degradation upon mechanical impact, e.g. impact by a stirring device. Mixing is however essential in order to achieve appreciable kinetics since diffusion control highly slows down reactions, especially enzymatic reactions. This implies that there is a strong need for efficient non-impact mixing processes. Whereas, this is quite easy to realize in a flow process, since the reaction mixture can be forced through a packed bed either by pressure or under-pressure, this is less evident in a batch process. Even in case flow would be used it is not evident to introduce other process steps as will be explained here below.

Another complexity in chemical and enzymatic reactions, especially in equilibrium reactions, resides in the fact that there is a need for withdrawal of at least one reaction product in order to shift the reaction to the right side of the reaction and in order to obtain an appreciable yield/conversion. Whereas in pure chemical reactions this can often be realized by precipitation, by extraction or by distillation (e.g. an azeotropic distillation) these procedures are very difficult in enzymatic reactions. In case precipitation would be used, there is a need to design the solvent or medium in such a way that at least one reaction product is insoluble. This will induce large restrictions on the use of the enzyme, since enzymes are very sensitive to solvent/medium effects (denaturation effect). The same holds for pH changes.

Also, distillation processes cannot be used since enzymes are very temperature sensitive and direct contact of enzyme and a gaseous phase is to be avoided. Specifically, bubbles originating at the surface may modify/denature the enzyme. Furthermore, excessive swelling may be observed in boiling conditions which may increase the mechanical sensitivity of the enzyme. There is thus again a clear need to other reaction product withdrawal methods using essentially low temperatures and no boiling conditions at the position of the enzyme as this would imply a too large enzyme/gas interaction.

It is clear from the above discussion that there is a need to solve both cited problems and there is especially a need to solve the problems in a way both problems can be handled at the same time and more preferably there is a need to find processes, devices and process set-ups to do this in a simple way, even more preferably in one single equipment, that will hereafter be called “body”.

The present invention addresses this need and offers embodiments that can indeed realize the optimized process in a simple set-up and even in one single “body”. However, the scope of the invention also comprises solutions with more than one equipment part since for specific reactions a “multi-body” embodiment can offer a better solution to the cited problems.

Further to their applications in equilibrium reactions, the devices and set-ups of the present invention are also highly suitable in situations where it is desirable to maintain a state of equilibrium. For example, the invention may find its application in situations where particular conditions are to be maintained, such as but not limited to maintaining a particular humidity in a room.

### SUMMARY OF THE INVENTION

In a first aspect, the present invention provides a device suitable for maintaining a state of equilibrium; said device comprising a container having a first zone (A) for holding a liquid formulation, the container being provided with a mechanism adapted to force the liquid formulation in a rotational movement in the container, wherein the container comprises a second zone (B) represented by a 3 dimensional structure having a top and a bottom, wherein the cross-section of the bottom of said structure is smaller than the cross-section of the top; the structure being arranged in the container such that the bottom points downward for being submerged in the liquid formulation, wherein in operation of the device, the liquid formulation is pushed upward from the bottom to the top of said structure, where the liquid formulation exits the structure and is returned to said first zone (A), and wherein said second zone (B) functions as a volatilization zone for volatizing and/or removing excess molecules from the liquid formulation, in order to maintain the state of equilibrium.

In a specific embodiment, said structure is a hollow funnel-shaped structure.

In yet a further embodiment of the present invention, said device further comprises a mechanism to rotate the structure around its own axis.

In a specific embodiment, said mechanism to force the liquid formulation in a rotational movement in the container and the structure are attached to the same shaft.

In yet a further embodiment, said structure is arranged in the container such that upon exiting the structure on the second side of the structure, the liquid formulation forms a thin film running downward on the inner wall of the container, thereby forming a further volatization zone (C) for volatizing and/or removing excess molecules from the liquid formulation.

In a different embodiment, the device of the present invention further comprises at least one inlet and at least one outlet means for a volatilization gas for volatizing and/or removing excess molecules from the liquid formulation in zone (B) and/or zone (C).

In yet a further embodiment, the device further comprises heating means to heat at least part of the device.

In a further aspect, the present invention provides the use of the device or process set-up as defined herein for maintaining a state of equilibrium in an enzymatic or chemical reaction, or for maintaining a particular relative humidity.

In a further aspect, the present invention provides a process set-up for equilibrium reactions comprising:
a) a reaction zone (A) comprising at least one non-volatile catalyst and a liquid reaction formulation comprising one or more reagents, wherein at least part of the liquid reaction formulation is intensively contacted with said catalyst without the use of mechanical impact;
b) a volatilization zone (B) being substantially free from catalyst, in which at least part of said liquid reaction formulation of step a) is contacted with an incoming unloaded gas, and reaction products from said reaction medium are volatized in said gas, thereby obtaining a loaded gas stream;
c) means to evacuate the loaded gas stream of step c) out of the volatilization zone (B);

In yet a further embodiment, the process set-up of the present invention further comprises:
d) means to evacuate at least part of the liquid reaction formulation from the reaction zone (A) to the volatilization zone (B), without substantially evacuating said catalyst; and
e) means to redirect at least part of the evacuated part of the liquid reaction formulation from the volatilization zone (B) to the reaction zone (A).

In a specific embodiment of the present invention said equilibrium reaction is a chemical or an enzymatic equilibrium reaction.

In yet a further embodiment, said equilibrium reaction is an enzymatic equilibrium reaction and said non-volatile catalyst is a non-volatile enzymatic catalyst; preferably a non-volatile heterogeneous enzymatic catalyst.

In a further embodiment, the process of the present invention is conducted at non-boiling conditions for the liquid reaction formulation.

In yet a further embodiment of the process set-up of the present invention, step b) comprises the formation of a thin film of at least part of said liquid reaction formulation.

In another embodiment of the process set-up of the present invention, step a) is performed by a centrifugally induced flow of the liquid reaction formulation through a bed of catalyst; or by rotating, tumbling or sliding the liquid reaction formulation and the at least one catalyst.

In yet a further embodiment of the present invention, said catalyst is immobilized to a surface, such as beads.

### BRIEF DESCRIPTION OF THE DRAWINGS

FIG. 1 shows an exemplary process set-up of the present invention representing a rotavapor-type of set-up comprising a rotating container.

FIG. 2 shows an exemplary process set-up of the present invention representing a rotating basket reactor set-up.

FIG. 3 shows an exemplary process set-up of the present invention representing a rotating bed reactor set-up.

FIG. 4 shows yet an exemplary process set-up of the present invention, wherein the rotating basket is acting as a pump.

FIG. 5 shows an exemplary process set-up of the present invention representing a continuous flow with incorporated gas sweeping set-up.

FIG. 6 shows an exemplary process set-up of the present invention including different separate compartments.

FIG. 7 shows an exemplary device according to the present invention suitable for performing an equilibrium reaction.

FIG. 8 shows the results of an experiment testing the reusability of the enzyme after performing up to 10 cycles of a process according to the invention.

FIG. 9 shows the results of an experiment testing the effects of boiling conditions on the enzymatic activity, under conditions of gentle agitation.

FIG. 10 shows the results of the impact of mechanical stirring in the case of applying gas sweeping on the enzymatic activity.

FIG. 11 shows the results of an enzymatic transesterification reaction making use of the set-up shown in FIG. 7.

### DETAILED DESCRIPTION OF THE INVENTION

The invention allows to cope with several technical problems associated with prior art devices and set-ups. Specific embodiments are possible where this can be realized in a multi body approach but also in a single body, the latter embodiment being preferred. This invention relates as well to batch as continuous flow processes and/or mixed processes. The invention allows to improve conversion and to improve recycling of a non-volatile (heterogenous) catalyst, thus to improve in general the industrial viability of manufacturing processes making use of the said processes. It is also very valuable in case of the synthesis of very temperature sensitive materials and/or reactive materials in general. The invention is very well suited for enzymatic reactions using immobilized enzymes since they are very sensitive to mechanical degradation upon stirring. It is also very useful for the realization of high conversions (approaching 100%) so that no further complex work-up after reaction is necessary.

The invention is particularly characterized by a combined process: step (1) non mechanical impacting mixing of reaction medium and immobilized catalysts (e.g. enzymes) AND step (2) the removal of a reaction product by gas purging and/or sweeping in a large gas/liquid contact zone and in the presence of catalysts (e.g. enzymes) and at temperatures lower than boiling point. Important is the introduction in the process approach of the creation of a large liquid/gas contact area (e.g. thin film zone) in the volatilization zone as well as the very specific operation condition of low temperature and absence of catalysts (e.g. enzymes) in that area.

Where the invention is particularly suitable in the context of equilibrium reactions, the devices and set-ups of the present invention are also highly suitable in situations where it is desirable to maintain a state of equilibrium. For example, the invention may find its application in situations where particular conditions are to be maintained, such as but not limited to maintaining a particular humidity in a room.

As already detailed herein above, in a first aspect, the present invention provides a device suitable for maintaining a state of equilibrium; said device comprising a container having a first zone (A) for holding a liquid formulation, the container being provided with a mechanism adapted to force the liquid formulation in a rotational movement in the container, wherein the container comprises a second zone (B) represented by a 3 dimensional structure having a top and a bottom, wherein the cross-section of the bottom of said structure is smaller than the cross-section of the top; the structure being arranged in the container such that the bottom points downward for being submerged in the liquid formulation, wherein in operation of the device, the liquid formulation is pushed upward from the bottom to the top of said structure, where the liquid formulation exits the structure and is returned to said first zone A, and wherein said second zone (B) functions as a volatilization zone for volatizing and/or removing excess molecules from the liquid formulation, in order to maintain the state of equilibrium.

In the same context, the present invention also provides a process set-up for equilibrium reactions comprising:
a) a reaction zone (A) comprising at least one non-volatile catalyst and a liquid reaction formulation comprising one or more reagents, wherein at least part of the liquid reaction formulation is intensively contacted with said catalyst without the use of mechanical impact;
b) a volatilization zone (B) being substantially free from catalyst, in which at least part of said liquid reaction formulation of step a) is contacted with an incoming unloaded gas, and reaction products from said reaction medium are volatized in said gas, thereby obtaining a loaded gas stream;
c) means to evacuate the loaded gas stream of step c) out of the volatilization zone (B).

Although said process set-up may be performed using any suitable type of device, it is advantageously performed using the devices as disclosed herein.

In the context of the present invention, the term “state of equilibrium” is meant to be a state of balance between different components. Accordingly in the context of the invention, this may be represented by a balance between components in the liquid formulation, either or not taking into account environmental conditions. For example, a state of equilibrium may be used in maintaining a particular humidity within a room, by applying a saturated liquid formulation (e.g. saturated salt formulation) to the device as disclosed herein. Alternatively, a state of equilibrium may also be maintained during an equilibrium reaction, such as a chemical or enzymatic reaction in which both the reagents (i.e. reactants) and reaction products of a liquid reaction formulation, are present in concentrations which have no further tendency to change within time, so that there is no observable change in the properties of the system. This state results when the forward reaction proceeds at the same rate as the reverse reaction. In order to drive such a reaction to the right side, i.e. towards the reaction products (forward reaction), it is desirable to extract reaction products from the reaction in order to temporarily break the equilibrium state, until the reaction finds equilibrium again. Continuous withdrawal of reaction products will accordingly keep on driving the equilibrium reaction to the right side, i.e. towards the formation of new reaction products.

As defined herein, the present invention uses a mechanism to force a liquid formulation in a rotational movement in the container. This feature has at least 2 purposes, first of all it allows to intimately mix the different components of the liquid formulation or liquid reaction formulation of the present invention, without inducing high (mechanical) impact on these components. In addition, it creates a flow of the liquid formulation or liquid reaction formulation, which is used as a transporting means between the different zones of the device or process set-up of the invention. For example, the rotational movement allows the liquid formulation or liquid reaction formulation to be pushed upward from the bottom (alternatively named first side) to the top (alternatively named second side) of the structure thereby creating a volatilization zone. However, the volatilization zone (B) may also be created in a process set-up wherein the rotational movement induces an upward movement of the liquid, or alternatively stated wherein the liquid formulation is forced, moved or pushed upward against the walls of the container.

Where in the context of the present invention, a structure is used, it is characterized in having 2 opposite sides, in particular a top and a bottom, wherein the cross-section of the bottom is smaller than the cross-section of the top, and wherein the smaller side (i.e. bottom) is being submerged in the liquid formulation or liquid reaction formulation. This way, the rotational movement pushes the liquid formulation or liquid reaction formulation from the smaller to the wider side of the structure, thereby creating a volatilization zone (B). To further assist in this upward movement, the structure itself may also be rotated. Accordingly in a further embodiment of the present invention, the device further comprises a mechanism to rotate the structure around its own axis.

Since, both the liquid formulation or liquid reaction formulation and the structure are preferably brought into a rotational movement, it is an advantage if these actions can be combined at once. Accordingly, in a specific embodiment, said mechanism to force the liquid formulation in a rotational movement in the container and the structure are attached to the same shaft.

In a specific embodiment, the structure may be a hollow funnel-shaped structure. However, any other structure suitable in the context of the present invention may also be used. In addition, the structure may comprise holes in the sidewalls, which allow the upward moving liquid formulation or liquid reaction formulation to exit the structure before it reaches the upper part thereof, thereby being able to control the area formed as the volatilization zone (B).

When exiting the structure at the second side (or top) of the structure or through one or more side-openings, the liquid formulation or liquid reaction formulation is returned back from the second zone (B) into the first zone (A), thereby potentially forming a further volatilization zone (C) on the outer walls of the structures or against the inner walls of the container, in case the structure is arranged closely to the inner walls.

Accordingly, in a further embodiment, said structure is arranged in the container such that upon exiting the structure on the second side (or top) of the structure, the liquid formulation forms a thin film running downward on the inner wall of the container, thereby forming a further volatization zone (C) for volatizing and/or removing excess molecules from the liquid formulation.

The eventual purpose of the present invention is to provide an as large as possible volatilization zone (B) and were applicable (C) allowing the efficient withdrawal of excess molecules in order to maintain a state of equilibrium or alternatively to drive an equilibrium reaction to the right side. This volatilization of excess molecules is done by means of a volatilization gas which picks up excess molecules and removes these from the device or process set-up of the present invention. Accordingly, the device and process-set-up of the present invention comprises inlet and outlet means for such volatilization gas, which is meant for volatizing and/or removing excess molecules from the liquid formulation or liquid reaction formulations in zone (B) and/or zone (C).

Since, heat may be beneficial in volatilization of excess molecules and/or driving a reaction, the invention further provides that the process set-up or devices as disclosed herein may additional comprise heating means. Accordingly, in a further embodiment, the device further comprises heating means to heat at least part of the device or process set-up.

In other words, the present invention provides a process set-up for equilibrium reactions with heterogeneous catalysis, comprising (1) a process zone (A), i.e. the reaction zone, where the reaction medium is intensively contacted with a heterogeneous catalyst(s) without the use of mechanical impact, (2) a second zone (B), i.e. the volatilization zone, where the reaction medium essentially free of catalyst is contacted over a large liquid/gas interface with an incoming unloaded gas so that at least one of the reaction products is removed by volatilization in said gas flow and by this is separated from the reaction mixture and (3) means to evacuate the loaded gas stream from zone (B) and a means of transporting the reaction medium back to zone (A), in this way allowing for a cyclic process up until the point where a selected conversion point is reached before discharging the reaction mixture from the process set-up, the whole process preferably being conducted at non-boiling condition for the reaction medium.

In the context of the present invention, the term “process set-up” (alternatively also named “system”) is meant to be any combination of reactors, tubes, pumps, stirrers, heaters, . . . i.e. hardware needed to perform the equilibrium reactions in accordance with the claims. In the following parts and drawings, different types of process set-ups and/or devices in accordance with the present invention are exemplified without being limited thereto.

In the context of the present invention, the term “reaction zone” is meant to be a part of the process set-up or device in which the equilibrium reaction takes place. This reaction zone may be a fully dedicated separate part (e.g. reactor) of the set-up or device, however, it could also form part of a more generic part of the set-up or device in which also other steps of the process are performed. The reaction zone is herein typically annotated as zone (A).

In the context of the present invention, the term “volatilization zone” is meant to be a part of the process set-up or device in which the loading of an incoming gas with excess molecules, such as reaction products takes place. This volatilization zone may be a fully dedicated separate part (e.g. reactor) of the set-up or device, however, it could also form part of a more generic part of the set-up or device in which also other steps of the process are performed. In the context of equilibrium reactions, the volatilization zone is substantially free from catalyst, meaning that it is either fully free from catalyst or comprising only minor amounts (e.g. trace amounts) of catalysts. This is important to ensure that no significant amount of catalyst is removed from the system, or is transferred into the volatilization zone, wherein the catalyst could potentially be inactivated. The volatilization zone is herein typically annotated as zone (B); or where applicable zone (C).

For example, a reactor may comprise a part in which the reaction takes place, while a different part of the same reactor serves the purpose of the volatilization zone. Accordingly, the reaction and volatilization zones may both be part of the same body within the reaction set-up, and detailed in some of the embodiments and drawings disclosed herein.

Alternatively, a volatilization zone may also be achieved by bubbling an incoming gas through the reaction medium or liquid formulation, thereby allowing any excess molecules or reaction products to be loaded into the gas.

In the context of the present invention, the term “heterogeneous catalyst” is meant to be a catalyst of which the phase differs from that of the reactants/reagents and reaction products.

Such phase distinction may for example rely on solid, liquid, or gas components, but may also rely on for example immiscible mixtures (e.g. oil and water). In the context of the present invention, the reactants/reagents are preferably in a solubilized state, whereas at least one reaction product is preferably a volatile component and the catalyst is preferably in a solid state. While the catalyst itself may be in a solid state, it may also be attached to a solid surface, such as beads, in order to obtain a kind of solid state. A catalyst in the context of the present invention is a component which increases the rate of a reaction but is in itself not consumed and thus preferably reusable.

In the context of the present invention, the term “reaction medium” is meant to be a composition comprising the reagents for the equilibrium reaction. Specifically preferred compositions are in liquid format such as a solution or dispersion in which the reagents are dissolved or dispersed respectively. The catalyst which is typically in solid format may also be dispersed in the reaction medium or may for example be contained in a container (e.g. basket or open container) which is accessible for the reaction medium.

In order to allow the equilibrium reaction to occur, it is of course necessary to contact the reaction medium with the catalyst. This contacting should be intensively in order to drive the reaction, however, not so roughly in that it may damage the catalyst. Especially for enzymatic catalysts, a soft treatment is advisable since these are easily damaged or detached from the surfaces to which they are attached. Accordingly, the contacting should be done in a way which does not use substantial mechanical impact, i.e. non-impact or impact-free contacting or at most low-impact contacting. Mechanical impact typically occurs if a high force or shock is applied over a short period of time when 2 or more substances collide. Such a force or shock typically has a greater effect than a lower force applied over a proportionally longer period of time. Accordingly, the contacting of the reaction medium with the catalyst is performed in such a way that the contacting is intensely enough to allow the reaction to occur, yet not resulting in damaging of the catalyst or in detaching the catalyst from the surface to which it is attached. In a specific embodiment of the present invention said equilibrium reaction is a chemical or an enzymatic equilibrium reaction.

In yet a further embodiment, said equilibrium reaction is an enzymatic equilibrium reaction and said non-volatile catalyst is a non-volatile enzymatic catalyst; preferably a non-volatile heterogeneous enzymatic catalyst.

In a particular embodiment, the contacting of the catalyst with the reaction medium is performed by a centrifugally induced flow of the reaction medium through a bed of catalyst; or by rotating, tumbling or sliding the reaction medium and the at least one catalyst.

In the context of the present invention the term “incoming unloaded gas” is meant to be a gas which is directed into the set-up and capable of being loaded with excess molecules or reaction products of the equilibrium reaction or liquid formulation. Evidently, when entering the set-up or devices of the invention, the gas should be unloaded, i.e. meaning that it does not substantially comprise molecules or reaction products equal to the ones to be loaded in the gas stream. By contacting at least part of the reaction medium with said gas in the volatilization zone, the excess molecules or reaction products are allowed to be loaded/volatized into the gas thereby obtaining a loaded gas stream. Subsequently, by removing the loaded gas stream from the system in the volatilization zone, part of the excess molecules or reaction products are removed from the system, thereby either maintaining a state of equilibrium or driving an equilibrium reaction to the right side in the reaction zone. Accordingly, the device and set-up of the present invention also provides means to evacuate the loaded gas stream out of the volatilization zone, and even preferably out of the set-up or device.

The process (i.e. reaction) is preferably conducted at non-volatizing conditions for the liquid formulation or liquid reaction formulation/medium itself, otherwise volatilization thereof may prematurely occur, whereas only volatilization of excess molecules or reaction products in the volatilization zone is desired. Said non-volatizing conditions depend on the type of formulations used, but are meant to be conditions which does not result in phase transition of liquid formulation or the reaction medium into the gas phase. While co-volatilization of the liquid formulation may occur to a limited extent, this may be compensated for by supplementing the process with liquid formulation during the process.

In a further embodiment of the present invention, the process set-up may further comprise:
d) means to evacuate at least part of the reaction medium from the reaction zone (A) to the volatilization zone (B), without substantially evacuating said catalyst; and
e) means to redirect at least part of the evacuated part of the reaction medium from the volatilization zone (B) to the reaction zone (A).

In principle, the set-up thus allows a more or less continuous flow of reaction medium from the reaction zone to the volatilization zone and back, wherein the catalyst remains in the reaction zone, and one or more of the reaction products may be extracted from the system in the volatilization zone.

In a preferred embodiment, the volatilization zone (B) comprises a thin film comprising part of the liquid formulation or liquid reaction formulation/medium. A thin film is a surface layer with a thickness of a few nanometers to micrometers. Within the context of the present invention, the formation of the thin film allows for the formation of a large liquid/gas contact area in the volatilization zone thereby allowing efficient withdrawal of excess molecules or reaction products from the set-up or device. Further, a thin film has the advantage to make evaporation over the total area of the film very easy since diffusion is reduced as retarding effect. Also a more stable temperature, more efficient energy use and less thermal stress on the reaction medium are further advantages of the thin film formation.

Several methods exist for making such a thin film, such as for example spin coating, dip coating, chemical vapor deposition, atomic layer deposition, . . . . In the context of the present invention the thin film is specifically formed by the rotational movement of at least part of the device or set-up. The thin film may thus for example be formed in the volatilization zone by slowly rotating the set-up thereby allowing the reaction medium to be pushed up against the walls of the system.

In a specific embodiment, the present invention provides a process set-up for equilibrium reactions comprising:
a) a reaction zone (A), wherein a reaction medium is intensively contacted with at least one heterogeneous catalyst without the use of mechanical impact;
b) means to evacuate the reaction medium of zone (A) to a volatilization zone (B), without substantially evacuating said catalyst;
c) a volatilization zone (B), wherein the reaction medium of step b) is contacted with an incoming unloaded gas, and reaction products in said evacuated reaction medium are volatized in said gas, thereby obtaining a loaded gas stream;
d) means to evacuate the loaded gas stream of step c) out of the volatilization zone (B);
e) means to redirect the reaction medium of step c) back to reaction zone (A);
f) optionally repeating steps a) to e); and
   wherein the process is conducted at non-boiling, or alternatively non-volatile conditions for the reaction medium.

### EMBODIMENTS OF THE INVENTION

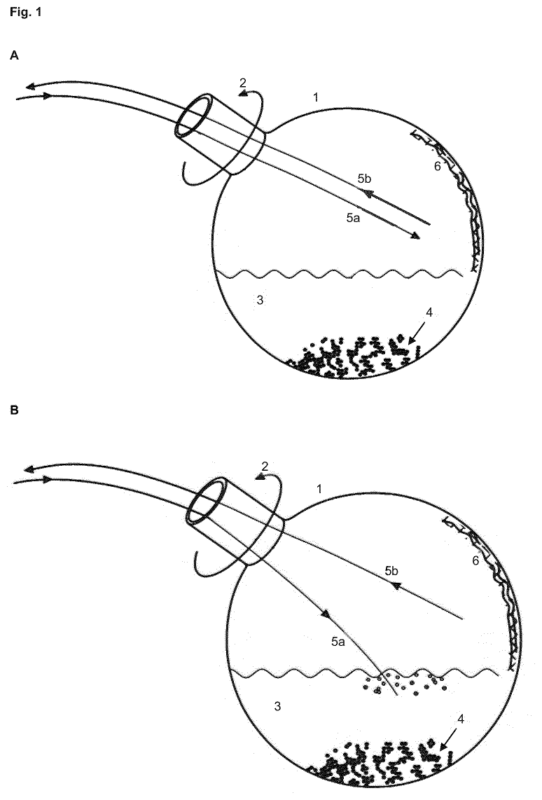

Some exemplary process set-ups and/or devices in accordance with the present invention are shown in FIGS. 1 to 6. These drawings are not limiting for the scope of the claims, and any other process set-up complying with the requirements of the claims is also encompassed herein.

FIG. 1 shows an exemplary process set-up of the present invention representing a rotavapor-type of set-up comprising a rotating container (1), in this case for example a flask. Due to the slowly rotating action-indicated by the rotating arrow (2)—of the container, the reaction medium (3) and catalysts (4) are intimately mixed allowing the reaction to occur. Yet, the rotating action is not vigorously, and as such the catalyst remains near the bottom of the container, due to gravity. Accordingly, a volatilization zone (B) may be realized in the top part of the reaction medium by an incoming gas stream (5a) in the upper non-liquid part of the container (FIG. 1A) or in the upper liquid part of the container (FIG. 1B), e.g. by bubbling in the incoming gas stream (unfilled circles). Since the catalyst mainly resides on the bottom of the container, it is not intimately contacted by the incoming gas stream. Yet, due to the rotating motion, reaction products formed at the bottom of the flask, where the catalyst resides, are still rather homogenously spread in the reaction medium. Accordingly, the incoming gas stream allows reaction products present in the top part of the reaction medium to be volatized and removed from the system, by an outgoing gas stream (5b). In addition, the rotating action of the container may allow the reaction medium to form a thin film (6) against the wall of the container above the liquid level of the reaction medium. As such, the incoming gas stream may further volatize any reaction products present in the thin film. The container may further be placed in a heater (not illustrated) such as a water bath which regulates the temperature needed in order for the reaction to occur, yet being low enough to not exceed the gas transition temperature of the reaction medium (i.e. below boiling point). In addition, in processes or states of equilibrium where the solvent and/or reagents may also be partially volatized, the container may be further supplemented with such solvent and/or reagent during operation, such as through a separate entrance port. This allows continuous or semi-continuous processes to be performed using the device of the present invention.

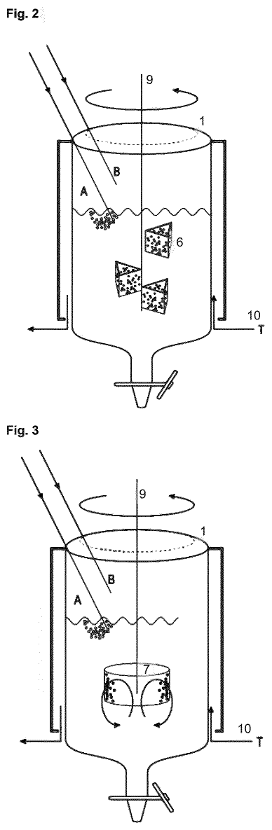

FIGS. 2 and 3 shows an alternative exemplary process set-up of the present invention representing a rotating basket (7) (FIG. 2) or bed (8) (FIG. 3) reactor set-up. Herein the container (1) itself is fixed, i.e. does not rotate. Yet, it may comprise a rotator (9) having fixed thereto one or more smaller containers (e.g. baskets) or a bed, containing the catalyst, which may for example be immobilized to a support. These containers/bed are preferably made from a material allowing fluids to pass through, such as by centrifugal forces. Such material may for example by a sieve-like or screen-like material used for building the wall of the 3D structure. Accordingly, due to the rotating movement of the rotator including these containers/bed, the reaction medium is passed through the walls of these containers/bed and along the catalyst, thereby allowing the reaction to occur. The reaction products can then again be removed from the system using similar options as illustrated for FIG. 1, i.e. bubbling of an incoming gas in the upper part of the reaction medium (i.e. being virtually free from catalyst) or allowing the gas to contact the upper surface of the reaction medium, and/or allowing the gas to contact any thin film formed to the walls of the container above the surface of the reaction medium. Heating of the reaction medium to the desired temperature may be performed by any suitable way, such as by heating (10) the walls of the container.

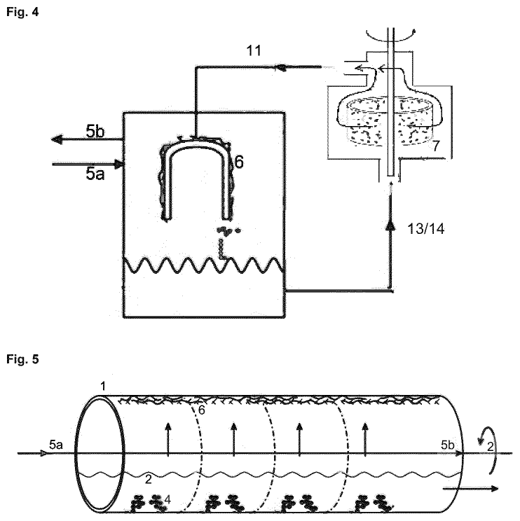

FIG. 4 shows yet a further exemplary process set-up of the present invention, wherein the rotating basket (7) is acting as a pump. Herein, the rotating action pushes the liquid upward in the center of the basket, after which it is forced sideward through the sieve-like basket. Hereafter the extracted liquid may be further processed such as by transporting (11) it to a second container (12) where it may form a thin film (6) allowing an incoming gas (5a) to volatize the reaction products, which are then removed from the system by the outgoing gas (5b). From the second container the remaining reaction medium can then be transported back (13) into the first container, such as by means of a pump (14).

FIG. 5 shows yet a further exemplary process set-up of the present invention representing a continuous flow with incorporated gas sweeping set-up. Herein, the container (1) is a longitudinal tube which may be rotating (2) along its longitudinal axis. Again, this rotating action is slowly enough the maintain the catalyst (4), which is for example immobilized to a support, at the bottom of the container. The reaction products can then again be removed from the system using similar options as illustrated for FIG. 1, i.e. bubbling of an incoming gas in the upper part of the reaction medium (i.e. being virtually free from catalyst) or allowing the gas to contact the upper surface of the reaction medium, and/or allowing the gas to contact any thin film formed to the walls of the container above the surface of the reaction medium. Heating of the reaction medium to the desired temperature may be performed by any suitable way, such as by heating the walls of the container. If desired the container may be placed in a slightly tilted manner, such as to create also a lateral movement of the reaction medium. By using this type of set-up, a continuous reaction system can be created.

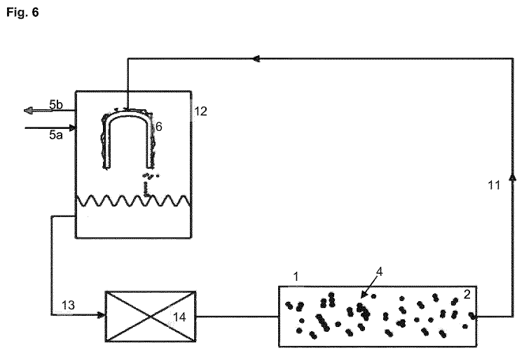

FIG. 6 shows yet a further exemplary process set-up of the present invention including different separate compartments. A first compartment is the reaction container (1), in the form of a packed column (i.e. a flow-like system), comprising the reaction medium (2) and catalyst (4), which may again be immobilized on a support. From this first compartment, the reaction medium being virtually free from catalyst, is transported (11) to a second container (12) where it may form a thin film (6) allowing an incoming gas (5a) to volatize the reaction products, which are then removed from the system by the outgoing gas (5b). From the second container the remaining reaction medium can then be transported back (13) into the first container, such as by means of a pump (14).

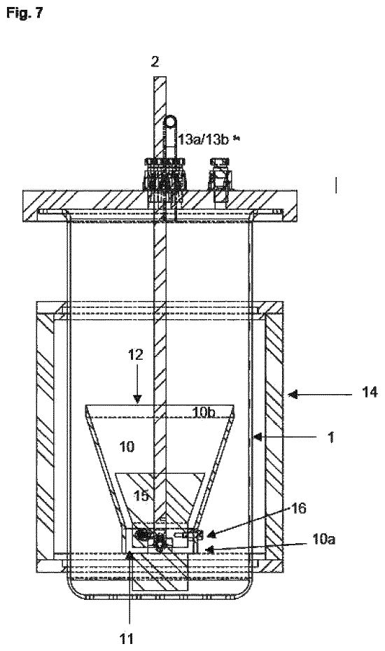

In a further aspect, the present invention provides a device suitable for performing an equilibrium reaction, such as an enzymatic equilibrium reaction. An exemplary device in the context of the invention is provided in FIG. 7. Said device comprises a reaction container (1) having a reaction zone A for holding a reaction medium and at least one catalyst. The container is provided with a shaft (2) which provides for the mechanism adapted to force the reaction medium in a rotational movement in the container. Herein said mechanism also drives the rotational movement of a hollow funnel-shaped structure (10) having 2 opposite sides. Wherein the cross-section of the bottom (10a) of said hollow structure is smaller than the cross-section of the top (10b), and the structure is arranged in the container such that the bottom points downward for being submerged in the reaction medium. The structure further comprises at least one opening arranged at the first side (11) and at least one opening (12) arranged at the second side of said structure. In operation of the device, by the rotational movement, the reaction medium enters the structure through the at least one opening at the first side (11), whereafter a thin film (volatilization zone B) of the reaction medium is pushed upward (by a force component resulting from the centrifugal force acting on the liquid) inside the hollow structure and exits the structure via the at least one opening at the second side (12). The device further comprises inlet (13a) and outlet means (13b) for a volatilization gas, allowing the removal of excess molecules or where applicable reaction products from the device. Finally, the device as disclosed herein also comprises a heating area (14) represented herein by a sleeve covering the outer surface of at least part of the device. In the present embodiment, the hollow structure comprises a further, yet smaller funnel-shaped structure (15), however, this is not essential in the context of the present invention, although it may provide for more stability of the structure during rotational movement thereof. However, if present, it should be designed as such that it does not substantially hinder the formation of a thin film on the inner surface of the main hollow structure (10). In the present case this is achieved by attaching the smaller funnel to the larger one by means of screws (16). In this way, the larger structure which provides for the thin film formation is not directly attached to the shaft, but indirectly by being attached to the smaller structure, which in turn is attached to said shaft.

### EXAMPLES

Very illustrative with respect to the above discussion on the complexity of enzymatic reactions is the preparation of sensitive acrylic esters. An example could be the preparation of the very temperature sensitive glycidyl acrylate. This could be envisaged by the transesterification of glycidol with methylacrylate. The resulting volatile reactant is in this case methanol. Another interesting example is the preparation of citronellyl acrylate by transesterification of citronellol with methylcrylate. Also interesting examples are e.g. acrylates starting from an acrylic moiety and high boiling alcohols, again using a transesterification of said alcohol with methylacrylate. Acrylic esters are very temperature sensitive. Especially when longer alcohols or polyols are used, the recovery of the pure ester is not possible by distillation as the temperature will induce polymerization of the acrylate. In the case of glycidyl acrylate also ring opening could occur as an additional unwanted reaction upon heating. The same holds for citronellyl acrylate as a shift of the double bond is possible at higher temperatures. In all cases cited above, this implies that the conversion towards the acrylic ester should be almost 100%. This implies on its turn an extreme shift in equilibrium to the reaction product side. This on its turn implies the need for a withdrawal of at least one reaction product, e.g. methanol in the case of said transesterifications with methylacrylate. In the case an esterification reaction of the alcohol with acrylic acid would be considered the volatile reaction product would be water.

In the classical approach, where a stirred reactor would be sued and e.g. an azeotropic distillation to withdraw the methanol or water, major problems arise. Stirring will promote degradation of the enzyme. The same azeotropic distillation induces boiling conditions, a condition that is disadvantageous for the enzyme as gas bubbles at the surface of the enzyme are noxious. The same high temperatures are also noxious for the enzyme since they will promote denaturation of the enzyme. On the other hand, the targeted 100% conversion, would imply long reaction time and hence a major danger to degrade the immobilized enzyme when impact stirring is used. Apart from thermal degradation of the enzyme as such, degradation of the envisaged acrylic compound as well as a degradation of the enzyme by reaction with the acrylic compound is possible at the higher temperatures. So there is the strong need to go for non-stirring conditions, a high evaporation rate and a large evaporation/volatization surface, preferably in combination, resulting in an improved enzymatic process to realize an industrial viable way of producing such sensitive acrylates.

Gas (Air) Flow with Controlled Relative Humidity (Water Vapor Pressure)

A rotavapor set-up was modified as shown in FIG. 1, the set-up being described herein above. A flask of 250 ml (diameter 8 cm) was attached to the rotavapor. The flask was partially (approximately 50%) immersed in a water bath at 25° C. The rotation speed of the flask was set at 120 rpm. A gas stream (5a) was lead into the set-up as shown in FIG. 1b. Dry air from a gas cylinder was fed to the set-up in order to produce humidified air. The flow rate was controlled by a mass flow controller purchased from Elneo (Wetteren, Belgium) and set at 400 ml/min. A stress test was made by feeding dry air and targeting the production of air with high relative humidity (RH). Two experiments were performed. In each experiment the flask was filled with 100 ml of a saturated salt solution, a saturated NaCl solution providing a theoretical equilibrium RH of 75% and a saturated KCl solution providing a theoretical equilibrium RH of 85%. The RH of the air flow leaving the flask (5b in FIG. 1) was measured using an electronic RH-sensor. The results are shown in table 1.

TABLE 1

| | T | RH th | RH 10 min | RH equi |
|---|---|---|---|---|
| NaCl sat | 22° C. | 75 | 60 | 68 |
| KCl sat | 22° C. | 85 | 65 | 74 |

Deviations in the equilibrium RH were smaller than 1% RH-units. Even using dry air, a fast humidification is observed and a high stable RH can be realized at a good flow rate using the object of the present invention. De equilibrium RH is lower than the theoretical value. This is due to the lower gas temperature, being the result of the expansion of the dry air from the pressurized gas cylinder.

Enzymatic Conversion at High Conversion with Reaction Product Withdrawal

The synthesis of citronellyl acrylate from citronellol and methyl acrylate was done using a transesterification reaction. The synthesis can be realized using an enzymatic process making use of the Candida Antartica CalB enzyme. The corresponding reaction equation is given as:

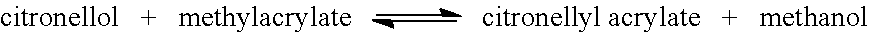

The equilibrium can be shifted to the right by removal of methanol from the reaction mixture. Use was made of an immobilized version of the said enzyme, in casu NOVOZYME 435 (Novozymes Denmark). Typical size of the immobilized enzyme is 100 μm. The transesterification was performed in n-hexane as a solvent medium.

Shifting the equilibrium towards the right implies efficient removal of methanol as a volatile component (b.p. 64.7° C.) from a solvent mixture comprising larger amounts of n-hexane with a close boiling point (69° C.). Dedicated experiments on methanol withdrawal from an n-hexane mixture in not boiling conditions were done and are described in examples 1-5 and comparative examples 6-9.

### Relevance of Combined Rotation and Volatilization

### Examples 1-5

A rotavapor set-up was modified as shown in FIG. 1, the set-up being described herein above above. A flask of 250 ml (diameter 8 cm) was attached to the rotavapor. The flask was partially (approximately 50%) immersed in a water bath at 50° C., a not boiling conditions for the system. The rotation speed of the flask was set at different rpm values (see table 2). An Argon gas stream (5a) was lead into the set-up as shown in FIG. 1. The Argon was delivered by a pressurized cylinder. The gas was expanded to atmosphere. The flow rate was controlled by a mass flow controller purchased from Elneo (Wetteren, Belgium) and set at 50 ml/min. Five experiments were performed. In each experiment the flask was filled with 100 ml of a mixture of n-Hexane containing 1 M methanol. The withdrawal of the methanol was monitored by taking small samples at different time intervals and analysing then using GC-FID analytical equipment. The loss of n-hexane through co-evaporation was compensated by adding fresh n-hexane at a 10 minutes interval. In the same way loss of mixture due to sampling was compensated. Different combination of sweeping, bubbling and rotation speed were explored. Results are shown in Table 2, line 1-5.

### Comparative Examples 6-9

In the same set-up as described in examples 1-5, comparative experiments 6-9 were done making use of conditions outside the patent description, i.e. with no rotation or no sweeping or no bubbling. Results are shown in Table 2, line 6-9.

TABLE 2

| | | | | Methanol (M) | | |
|---|---|---|---|---|---|---|
| | Rotation | | | 0 | 10 | 20 |
| Experiments | (rpm) | Sweeping | Bubbling | min | min | min |
| Example 1 | 30 | X | 0 | 1.00 | 0.45 | 0.10 |
| Example 2 | 76 | X | 0 | 1.00 | 0.27 | 0.04 |
| Example 3 | 136 | X | 0 | 1.00 | 0.15 | 0.03 |
| Example 4 | 201 | X | 0 | 1.00 | 0.15 | 0.02 |
| Example 5 | 201 | 0 | X | 1.00 | 0.18 | 0.02 |
| Comparative 1 | 0 | 0 | 0 | 1.00 | 0.77 | 0.77 |
| Comparative 2 | 201 | 0 | 0 | 1.00 | 0.71 | 0.66 |
| Comparative 3 | 0 | X | 0 | 1.00 | 0.71 | 0.57 |
| Comparative 4 | 0 | 0 | X | 1.00 | 0.76 | 0.52 |

From Table 2 it can clearly be seen that combining rotation and sweeping and/or bubbling (thereby allowing volatization) leads to a high efficiency in methanol withdrawal. It can be seen that already slow rotation gives a marked improvement. It can also be seen from the table that high rotation speeds leads only to minor improvements over low rotation speeds.

Enzymatic transesterification reaction according to the present invention and also comparative experiments were done using the same set-up and are described in the examples 10 to 13.

### Example 10

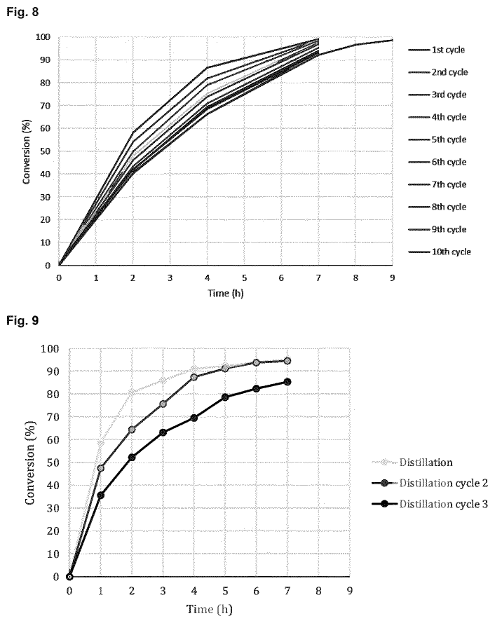

Citronellyl acrylate was synthesized using the enzymatic transesterification reaction as outlined above. 100 ml of a substrate solution containing 1M citronellol and 2M methyl acrylate in hexane were prepared. 2 g of Novozyme 435 were added for 100 ml of substrate solution to obtain a concentration of 20 g/l. Methyl acrylate and hexane which were co-evaporated were replenished during the experiment to keep the concentrations and volume in line with the reaction progress. The set-up in FIG. 1b was used. Temperature was set at 50° C., i.e. non boiling conditions for the reaction mixture. Rotation speed 70 rpm. Flow rate of Ar 50 ml/min. Successive cycles, in casu up to 10, were run in order to observe the reusability of the enzyme. After each reaction the enzyme was removed by decantation, washed with n-hexane, washed with acetone and eventually rewashed with n-hexane and used without drying. Results are shown in FIG. 8.

From FIG. 8 it can be seen that the enzyme can be reused easily up to 10 cycles. After a small decline in efficiency due to washing out of not firmly bound enzyme, a steady state can be realised, implying that the reaction conditions are not detrimental to the enzymatic activity. Also a high conversion (>95%) can be realized in a time frame of less then 8 hours, being a daily shift, making this method economically viable.

### Comparative Example 11

Example 10 was repeated in set-up FIG. 1, however the temperature was set at 70° C., corresponding to boiling conditions of the system. No gas bubbling nor sweeping was applied. Results are given in FIG. 9. From FIG. 9, it can be seen that despite the soft agitation (rotating flask) the boiling conditions are indeed causing deterioration of the enzymatic activity, as explained herein above. This will limit the reusability of the enzyme and thus will lead to a not economically viable production process. Low temperature (in particular non-volatile conditions) and soft agitation are important as can be deduced from example 10.

### Comparative Example 12

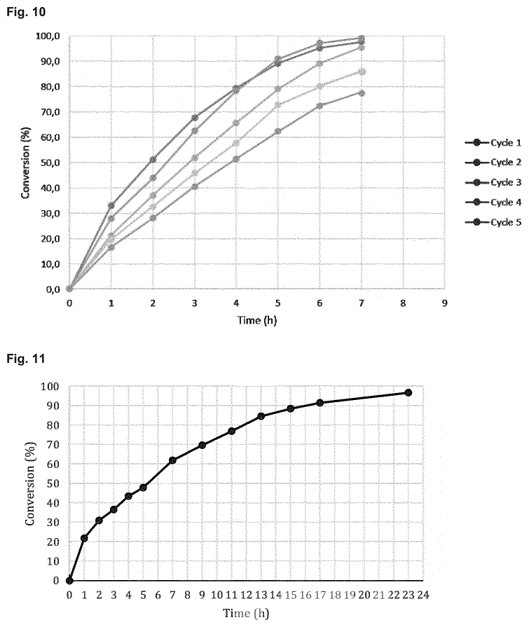

An enzymatic transesterification experiment was done in a way as described in example 10. The difference is that no rotavap modified set-up was used and agitation in the flask was done by means of a mechanical stirrer at 200 rpm. Further conditions are: 50° C.—Argon sweeping 50 ml/min. Results are shown in FIG. 10. From FIG. 10 it can be seen that mechanical stirring even in the case of applying gas sweeping does not result in a process where the enzymatic activity is preserved. Mechanical impact is detrimental to the enzyme as argued above. This will limit the reusability of the enzyme and thus will lead to a not economically viable production process. Low temperature and soft agitation are important as can be deduced from example 10.

### Example 13

The enzymatic transesterification reaction described in example 10 was repeated however making use of the set-up shown in FIG. 7. This set-up allows for higher volumes to be produced. The diameter of the reactor is 150 mm with a height of 300 mm. The funnel has a top diameter of 120 mm and a bottom diameter of 55 mm. The total height of the funnel is 100 mm. The enzyme was loaded in a basket mounted underneath the funnel as shown in FIG. 7. The volume of the reaction mixture was set at 1,5l. Rotation was set at 400 rpm and an Argon gas stream was applied of 50 ml/min in a sweeping mode. The gas inlet was positioned between the funnel and the reactor wall, approximately 1.5 cm above the surface of the reaction mixture. In order to reduce the cost of the enzyme a lower amount was used, in case 9,7 g resulting in a 3-fold lower concentration in comparison to examples 1-5.

For the sake of comparison example 1-5 should be compared to example 12 by expressing conversion/hour. enzyme. Co-evaporation of hexane and methyl acrylate was compensated for. The result is given in FIG. 11. From FIG. 11 it is seen that a conversion of >95% is realized in a time frame of 21 hours, being identical in conversion rate to the conversion in example 10. The time frame in FIG. 11 is 3 times larger due to the 3-fold lower enzyme loading. This confirms the functionality of the embodiment 7 of the present invention. This embodiment is very suited for scaling-up as explained herein above.

1. A device suitable for maintaining a state of equilibrium; said device comprising a container having a first zone (A) for holding a liquid formulation, the container being provided with a mechanism adapted to force the liquid formulation in a rotational movement in the container, wherein the container comprises a second zone (B) represented by a 3 dimensional structure having a top and a bottom, wherein the cross-section of the bottom of said structure is smaller than the cross-section of the top; the structure being arranged in the container such that the bottom points downward for being submerged in the liquid formulation, wherein in operation of the device, the liquid formulation is pushed upward from the bottom to the top of said structure, where the liquid formulation exits the structure and is returned to said first zone A, and wherein said second zone (B) functions as a volatilization zone for volatizing and/or removing excess molecules from the liquid formulation, in order to maintain the state of equilibrium.

2. The device according to claim 1, wherein the structure is a hollow funnel-shaped structure.

3. The device according to anyone of claim 1 or 2, further comprising a mechanism to rotate the structure around its own axis.

4. The device according to anyone of claims 1 to 3, wherein the mechanism to force the liquid formulation in a rotational movement in the container and the structure are attached to the same shaft.

5. The device according to anyone of claims 1 to 4, wherein the structure is arranged in the container such that upon exiting the structure on the second side of the structure, the liquid formulation forms a thin film running downward on the inner wall of the container, thereby forming a further volatization zone (C) for volatizing and/or removing excess molecules from the liquid formulation.

6. The device according to anyone of claims 1 to 5 further comprising at least one inlet and at least one outlet means for a volatilization gas for volatizing and/or removing excess molecules from the liquid formulation in zone (B) and/or zone (C); in particular by introducing the volatilization gas such that it flows across the liquid formulation or bubbles in the liquid formulation.

7. The device according to anyone of claims 1 to 6, wherein the device further comprises heating means to heat at least part of the device.

8. Use of the device according to anyone of claims 1 to 7 for maintaining a state of equilibrium in an enzymatic or chemical reaction, or for maintaining a particular relative humidity.

9. A process set-up for equilibrium reactions comprising:
a) a reaction zone (A) comprising at least one non-volatile catalyst and a liquid reaction formulation comprising one or more reagents, wherein at least part of the liquid reaction formulation is intensively contacted with said catalyst without the use of mechanical impact;
b) a volatilization zone (B) being substantially free from catalyst, in which at least part of said liquid reaction formulation of step a) is contacted with an incoming unloaded gas, and reaction products from said reaction medium are volatized in said gas, thereby obtaining a loaded gas stream;
c) means to evacuate the loaded gas stream of step c) out of the volatilization zone (B);

10. The process set-up of claim 9 further comprising:
d) means to evacuate at least part of the liquid reaction formulation from the reaction zone (A) to the volatilization zone (B), without substantially evacuating said catalyst; and
e) means to redirect at least part of the evacuated part of the liquid reaction formulation from the volatilization zone (B) to the reaction zone (A).

11. The process set-up of anyone of claims 8-10; wherein said equilibrium reaction is a chemical or an enzymatic equilibrium reaction; and wherein said non-volatile catalyst is a non-volatile chemical or enzymatic catalyst; preferably a non-volatile heterogeneous chemical or enzymatic catalyst.

12. The process set-up of claim 11; wherein said process is conducted at non-boiling conditions for the liquid reaction formulation.

13. The process set-up of anyone of claims 8 to 12, wherein step b) comprises the formation of a thin film of at least part of said liquid reaction formulation.

14. The process set-up of anyone of claims 8 to 13, wherein step a) is performed by a centrifugally induced flow of the liquid reaction formulation through a bed of catalyst; or by rotating, tumbling or sliding the liquid reaction formulation and the at least one catalyst.

15. The process set-up of anyone of claims 8 to 14, wherein said catalyst is immobilized to a surface, such as beads.
